(19) United States
(12) Patent Application Publication
(10) Pub. No.: US 2010/0130839 A1
(43) Pub. Date: May 27, 2010

(54) IMPLANTABLE DEVICES AND METHOD FOR DETERMINING A CONCENTRATION OF A SUBSTANCE AND/OR MOLECULE IN BLOOD OR TISSUE OF A PATIENT

(75) Inventor: Kenneth Dowling, Bro (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St. Jude Medical AB

(21) Appl. No.: 12/596,871

(22) PCT Filed: Apr. 27, 2007
(86) PCT No.: PCT/SE2007/000410

§ 371 Date: Oct. 21, 2009

(51) Int. Cl. A61B 5/1459

(52) U.S. Cl. 600/316; 600/341; 600/339

(57) ABSTRACT

In a medical lead having a sensor, an implantable device connectable to such a medical lead and a method for determining the concentration of a substance and/or molecule in blood or tissue of a patient, in particular for determining the concentration of substances and or molecules such as oxygen, carbon dioxide, molecules representative of a PH value, glucose, urea, ammonia, lactose, hormones and insulin, photoluminescent molecules embedded in a carrier, in which the substance and/or molecule to be analyzed diffuse, are excited to emit light by light emitted by a light source of the sensor. The determination of the concentration of the substance and/or molecule is based on the characteristics of the light emitted by the photoluminescent molecules.

### BACKGROUND OF THE INVENTION

1. Field of the Invention

The present invention generally relates to the field of implantable medical devices. More specifically, the present invention relates to a sensor, a medical lead, an implantable device and a method for determining the concentration of a substance and/or molecule in blood or tissue of a patient, in particular for determining the concentration of substances and or molecules such as oxygen, carbon dioxide, molecules representative of a PH value, glucose, urea, ammonia, lactose, hormones and insulin.

2. Description of the Prior Art

Many substances and/or molecules are comprised in blood, which works as a transporting medium to either supply these substances and/or molecules to organs of a human body or to remove them. As examples, blood can supply molecules such as oxygen or nutrients such as glucose and amino acids to tissues of organs while it can also remove substances such as urea and carbon dioxide from these organs. The concentrations of these substances and/or molecules in blood or tissue of a human body determine the functioning of the organs and therefore the health of the human body. For instance, the content of oxygen in blood is an important parameter to determine for understanding cardiac and pulmonary function of a patient and is particularly important to monitor for adjusting the frequency of a pacemaker.

Conventional sensors employed to monitor the content of oxygen in blood are based on photo-spectrometric properties of blood, which properties depend on the oxygenation level of blood. In these sensors, such as the sensor disclosed in U.S. Pat. No. 4,815,469, a light emitting diode is used to illuminate the blood of a patient and a photo-detector is used to detect the light reflected by the blood, the light emitting diode and the photo-detector being separated from the blood by means of an interface, e.g. being embedded in a medical pacing lead. The analysis of the spectrum of the reflected light then results in the content of oxygen in the blood. An alternative to such a measurement is a measurement based on transmitted light where the light source and the photo-detector are positioned on either side of the blood specimen to be analyzed and where the light absorption in hemoglobin is used as an indicator representative of the content of oxygen in blood. One of the requirements for detectors based on reflectance and absorbance measurements is that the interface between the light source and the blood and the interface between the photo-detector and the blood must be unimpeded in the wavelengths used for analysis. However, phenomena such as thrombosis, tissue growth or impeding of blood flow at these interfaces are common and prevent accurate and continuous measurements of the oxygen content of blood such as would be required for pacing or other cardiac therapy. In addition, these optical techniques enhance these phenomena, which cause the measurements to be more and more difficult to implement with time.

Further, another problem with conventional reflectance and absorbance measurements is that the result of the measurement is dependent on variable parameters such as blood flow rate, red blood cell count, white blood cell count, platelet count and other variable factors in the bloodstream since the measurement directly involves the constituents of the blood. In addition, the detectors based on reflectance and absorbance require up to three wavelengths, which is expensive and complicates the detector in terms of design and space management. Thus, there is a need for providing improved methods and devices that would overcome at least some of these problems.

### SUMMARY OF THE INVENTION

An object of the present invention is to wholly or partly overcome the above disadvantages and drawbacks of the prior art and to provide an improved alternative to the above techniques and prior art.

A further object of the present invention to provide an improved sensor, a medical lead comprising such a sensor, an implantable device connectable to such a medical lead and an improved method for determining the concentration of a molecule and/or a substance such as oxygen, carbon dioxide, molecules representative of a PH value, glucose, urea, ammonia, lactose, hormones and insulin in blood or tissue of a patient that wholly or partly overcome the above mentioned problems.

Another object of the present invention is to provide an improved sensor that provides accurate and repeatable measurements of a concentration of a molecule and/or a substance such as oxygen, carbon dioxide, molecules representative of a PH value, glucose, urea, ammonia, lactose, hormones and insulin in blood or tissue of a patient.

Hence, according to a first aspect of the present invention, an implantable sensor for determining a concentration of a substance and/or molecule in blood or tissue of a patient is provided. The sensor includes at least one light source adapted to, during measurement sessions, emit light at least at a first wavelength, at least one photo-detector and at least one type of photoluminescent molecules embedded in a carrier selectively permeable to the substance and/or molecule. A part of the carrier is in contact with a region of the patient, which region comprises the substance and/or molecule, and the light source and the photo-detector are optically connected to the carrier. The photoluminescent molecules emit light in response to excitation by the light emitted from the light source, and the substance and/or molecule reacts with the photoluminescent molecules to alter characteristics of the light emitted from the photoluminescent molecules. The photo-detector is adapted to detect the light emitted from the photoluminescent molecules and to provide output signals representative of the characteristics to determine the concentration of the substance and/or molecule.

According to a second aspect of the present invention, a medical lead embodies an implantable sensor such as the improved sensor described above. The inventive medical lead may include the same additional features as the features that will be described below for the implantable sensor, thus providing similar advantages.

According to a third aspect of the present invention, an implantable device connectable to a medical lead embodies an implantable sensor such as the improved sensor described above is provided. The implantable device further has a blood constituent determining device adapted to receive output signals from the photo-detector in order to determine, based on the received signals, the concentration of the molecules and/or substances being comprised in the blood composition or the tissue of a patient. The inventive implantable device may comprise the same additional features as the features that will be described below for the implantable sensor, thus providing similar advantages.

According to a fourth aspect of the present invention, a method for determining a concentration of a substance and/or a molecule in blood or tissue of a patient is provided. This method includes a first step of, during measurements sessions, exciting photoluminescent molecules embedded in a carrier selectively permeable to the substance and/or molecule such that the photoluminescent molecules emit light. Further, the method includes s a step of detecting the light emitted from the photoluminescent molecules, and a step of determining the concentration of the substance and/or molecule based on characteristics of the detected light.

The present invention is based on the insight that photoluminescent molecules can be arranged in an implantable device to determine the concentration of a substance and/or a molecule in blood or tissue of a patient.

The inventive sensor described above is advantageous in that it enables real-time measurements over long time periods. The sensor according to the present invention discloses a low sensitivity to phenomena such as thrombosis, tissue growth or impeding of blood flow since the interface between the sensor and the region to be analyzed is not illuminated, as opposed to prior art sensors in which such phenomena are enhanced by the fact that light has to pass through the interface between the sensor and the blood. Further, as the optical part of the measurement does not take place directly in the blood or the tissue of the patient but in the carrier, the determination of the concentration is not dependent on variable parameters such as blood flow rate, red blood cell count, white blood cell count, platelet count and other variable factors in the bloodstream. Further, as the molecules to be analyzed can penetrate tissue of organs, the sensor may either be arranged directly in contact with the blood of a patient or in the tissue of a patient. In addition, the sensor can be made very compact and cheap.

In another embodiment, the implantable sensor may have an optical filter arranged at the photo-detector to selectively transmit the light emitted by the photoluminescent molecules to the photo-detector, which is advantageous since the amount of light directly received from the light source by the photo-detector is minimized.

In further embodiments, the carrier of the implantable sensor may be made of a material comprised in the group of silicone rubber (also known as polydimethylsiloxane), organosubstitute silicones such as poly(R—R′-siloxane) wherein R and R′ are one of the following {methyl, ethyl, propyl, butyl, “alkyl”, “aryl”, phenyl and “vinyl”} and not necessarily equal to each other, polyurethane, poly(1-trimethylsily-1-propyne), polystyrene, poly(methylmethacrylate), poly(vinyl chloride), poly(isobutylmethacrylate), poly(2,2,2-trifluoroethylmethacrylate), ethylcellulose, cellulose acetobutyrate, cellulose acetate, gas-permeable polytetrafluoroethylene and thermoplastic polyurethanes and copolymers.

In another embodiment, the light source of the implantable sensor may be a solid state light source, preferably a light emitting diode, which is advantageous since light emitting diodes are small and therefore easily incorporated in the sensor, thus alleviating the requirement on space issues close to the region to be analyzed. Further, the use of light emitting diodes is advantageous since they have a rather long lifetime as compared to most common light sources. Further, light emitting diodes can be selected so that the level of infrared radiation contained in their output power spectra is low in order to minimize the generation of heat by the radiation. In a particular embodiment, the light source may be a light-emitting diode adapted to emit light in the range of 390-1650 nm.

In another embodiment, the photo-detector of the implantable sensor may be a solid state photo-detector, which is advantageous since such detectors present excellent linearity with respect to incident light, have low internal noise, wide spectral response, long life time, and are mechanically resistant, compact and lightweight. In particular embodiments, the photo-detector shall be sensitive in the range of 350-1800 nm, preferably in the range of 350-800 nm, which is advantageous since these are ranges within which most common photoluminescent molecules emit light. In particular embodiments, the photo-detector may be one of the group comprised of a pn photodiode, a pin photodiode, a Schottky photodiode, an avalanche photodiode, a silicon photodiode, a planar InGaAs photodiode, a SiGe-based optoelectronic circuit and a InGaN/GaN multiple quantum well pn junction.

In an embodiment, the implantable sensor of the present invention may be adapted to determine the concentration of any type of substances and/or molecules being comprised in the blood composition or in the tissue of a patient. In an embodiment, the sensor is adapted to determine the saturation or the concentration of oxygen in the region at which the carrier is arranged. In another embodiment, the sensor is adapted to determine the concentration of carbon dioxide in the region at which the carrier is arranged. In yet another embodiment, the sensor is adapted to determine the pH-value in the region at which the carrier is arranged. In other embodiments, the sensor is adapted to determine the concentration of a substance and/or a molecule of anyone of the group of glucose, urea, ammonia, lactose, hormones or insulin, which molecule or substance is contained in the region at which the carrier is arranged.

In further embodiments, the photoluminescent molecules may be fluorescent molecules or phosphorescent molecules. Although it would be preferable to use fluorescent molecules as they are generally more stable over time, it is preferable to use phosphorescent molecules as phosphorescence leads to longer timescale, which then facilitates the design of the electronics in the sensor.

In further embodiments, the photoluminescent molecules used in the implantable sensor of the present invention may be one of the group comprised of pyrene, a pyrene derivative (vinylpyrene, methoxypyrene), a polyaromatic carrier, an ionic probe (ruthenium trisbypyridine) and Pd-tetra (4-carboxyphenyl) benzoporphin (Oxyphor). In particular embodiments, the concentration of these photoluminescent molecules is comprised in the range of 20 ppb to 2% w/w, preferentially in the range of 0.1-500 ppm, and more preferentially at approximately 5 ppm.

In further embodiments, the inventive implantable sensor may be arranged at the heart of a patient, in arterial blood, in venous blood or in the tissue of a blood-containing organ of a patient. Thus, many arrangements are possible, the requirement being that the carrier is in contact with a region of the patient comprising the molecules and/or substances to be analyzed such that these molecules and/or substances can react with the photoluminescent molecules.

In another embodiment, the light source and the photo-detector are hermetically isolated from the region of the patient.

In further embodiments, one of the characteristics of the light emitted by the photoluminescent molecules, which light may be altered by the reaction between the molecules and/or substances to be analyzed and the photoluminescent molecules, may be the intensity, the lifetime or the wavelength. This is advantageous since this provides different manners of determining the concentration of the molecules and/or substances to be analyzed. In a particular embodiment, it may be advantageous to use the lifetime of the light rather than its intensity since any change in the concentration of the photoluminescent molecules with time will affect the intensity but not the lifetime. Such changes in concentration are possible because of various biological processes.

In an embodiment, the implantable device may have an amplifier for amplifying the output signals sent from the photo-detector to the blood constituent determining device, which is advantageous since the output signals of the photo-detector may, in some cases, be weak.

In further embodiments, the lifetime of the light emitted by the photoluminescent molecules may be determined according to different types of measurements. In an embodiment, the implantable device may comprise a pulse generating means connected to the light source to cause the light source to emit light pulses such that the blood constituent determining device, which is adapted to control the photo-detector and the pulse generating means, can perform time resolved measurements for determination of the lifetime. In another embodiment, the implantable device may include a frequency modulating means connected to the light source to modulate the light emitted from the light source such that the blood constituent determining device, which is adapted to control the photo-detector and the frequency modulating means, can perform frequency modulation measurements for determination of the lifetime.

All references to “a/an/the [element, device, component, means, step, etc]” are to be interpreted as referring to at least one instance of the element, device, component, means, step, etc., unless explicitly stated otherwise. Further objectives of, features of, and advantages with, the present invention will become apparent when studying the following detailed disclosure, the drawings and the appended claims. Those skilled in the art will realize that different features of the present invention can be combined to create embodiments other than those described in the following.

### BRIEF DESCRIPTION OF THE DRAWINGS

FIG. 1 shows an implantable sensor according to a first aspect of the present invention.

FIG. 2 shows a medical lead according to a second aspect of the present invention.

FIG. 3 shows an implantable device according to a third aspect of the present invention.

FIG. 4 shows a block diagram of a method for determining a concentration of a substance and/or molecule in blood or tissue of a patient according to a fourth aspect of the present invention.

All the figures are schematic, not necessarily to scale, and generally only show parts which are necessary in order to elucidate the invention, wherein other parts may be omitted or merely suggested.

### DESCRIPTION OF THE PREFERRED EMBODIMENTS

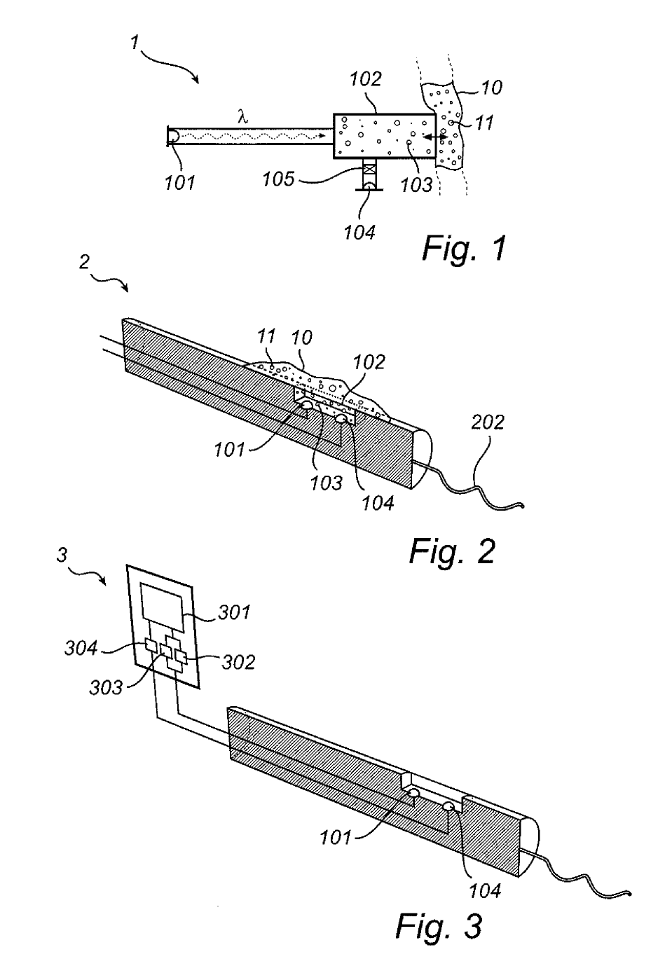

With reference to FIG. 1, a first aspect of the present invention will be described below.

FIG. 1 shows an implantable sensor according to an embodiment of the present invention. The sensor 1 comprising a carrier 102 in which photoluminescent molecules 103 are embedded. The carrier 102 is partially in contact with a region 10 of a patient in which the molecules and/or substances 11 are present for which the concentration shall be determined. The sensor 1 further comprises a light source 101 and a photo-detector 104 which are optically connected to the carrier 102 such that the light emitted from the light source 101 can excite the photoluminescent molecules 103 and such that the light emitted from the photoluminescent molecules 103 in response to this excitation can be detected by the photo-detector 104. The carrier 102 is selectively permeable to the molecules and/or substances 11 for which the concentration has to be determined such that these molecules 11 can diffuse from the region 10 of the patient into the carrier 102. The photoluminescent molecules 103 are selected to react with the molecules and/or substances 11 coming from the region 10 in such a manner that the characteristics of the light emitted from the photoluminescent molecules 103 is altered because of the reaction. Depending on the concentration of the molecules and/or substances 11 in the region 10 of the patient, the characteristics of the light emitted from the photoluminescent molecules 103 will then be altered to different degrees. Thus, a determination of the concentration of the molecules 11 in the region 10 of the patient is enabled.

In an embodiment, the light source 101 and the photo-detector 104 are directly arranged at a side of the carrier 102 or in the carrier 102. In another embodiment, the light source 101 and the photo-detector 104 are optically connected to the photoluminescent molecules 103 comprised in the carrier 102 by means of optical fibers. The optical fibers may be arranged between the light source 101 and the photoluminescent molecules 103 and/or between the photo-detector 104 and the photoluminescent molecules 103, which means that the light source 101 and the photo-detector 104 can be arranged at a certain distance from the analyzed region 10. The optical fibers are used to guide light from the light source and to guide light to the photo-detector, respectively. Using optical fibers to connect the light source 101 and the photo-detector 104 enables to reduce the size of the part of the sensor 1 that has to be placed in contact with the region 10 of the patient, which is advantageous if the space at the region 10 of the patient is limited.

In an embodiment, the light source 101 and the photo-detector 104 are fabricated on a same substrate using e.g. standard semiconductor technology. The carrier 102 is then made of a material, such as a silicon adhesive, like e.g. the commercially available Rehau RAU-SIK SI-1511, containing the photoluminescent molecules and spread over the substrate. The substrate can then be used as a support for the light source 101, the photo-detector 104 and the carrier 102. Such a substrate would also enable easy connection between the light source 101, the photo-detector 104 and other components used to analyze the signal output of the photo-detector 104. The thickness of the adhesive film containing the photoluminescent molecules would typically be comprised between 0.03 and 3 mm.

In yet a further embodiment, the light source 101 and the photo-detector 104 can be fabricated on two separate substrates and joined together by means of the carrier 102, e.g. the silicon adhesive mentioned above, containing the photoluminescent molecules 103.

In an embodiment, a filter 105 may be arranged in front of the photo-detector 104 to select the range of wavelength that shall be transmitted to the photo-detector. In particular, the filter 105 is used to eliminate the part of the excitation light emitted by the light source 102 that is reflected by the photoluminescent molecules 103 or by the carrier or carrier matrix 102.

In further embodiments, the carrier 102 can be made as a material comprised in the group of silicone rubber (also known as polydimethylsiloxane), organosubstitute silicones such as poly(R—R′-siloxane) wherein R and R′ are one of the following {methyl, ethyl, propyl, butyl, “alkyl”, “aryl”, phenyl and “vinyl”} and not necessarily equal to each other, polyurethane, poly(1-trimethylsily-1-propyne), polystyrene, poly(methylmethacrylate), polyvinyl chloride), poly(isobutylmethacrylate), poly(2,2,2-trifluoroethylmethacrylate), ethylcellulose, cellulose acetobutyrate, cellulose acetate, gas-permeable polytetrafluoroethylene and thermoplastic polyurethanes and copolymers, in which material the photoluminescent molecules 103 are embedded. Further, these materials, in particular silicone rubber, thermoplastic polyurethanes and copolymers, gas-permeable polytetrafluoroethylene, are well adapted in the present invention since they are implantable biomaterials. In a particular embodiment, silicone rubber is well adapted if the sensor 1 is used to determine the concentration of oxygen in the region 10 since its permeability to oxygen is about ten times higher than in most polymeric materials, namely 6.95×10¹¹ cm⁻²s⁻¹Pa⁻¹, which corresponds to a diffusion coefficient of about 1.45×10⁻⁵ cm²s⁻¹. Thus, the diffusion of oxygen into silicone rubber will be rapid enough for measuring fast changes in the oxygenation of the region of the patient. However, other materials than silicone rubber and polyurethane may be used, such as polystyrene, the requirement being that the diffusion coefficient of the material for oxygen shall be sufficient to sense the changes in the oxygenation of the region 10. The high oxygen permeability of silicone rubber allows high flexibility on the design. Thus, a thick layer of silicone rubber could be used. In addition, the sensor 1 would remain sufficiently sensitive even if some limited amount of tissue overgrowth occurred at the side of the carrier 102 in contact with the region 10 of the patient.

The light source 101 may be a solid state light source, preferentially a light emitting diode, which is advantageous since light emitting diodes are small and can therefore easily be incorporated in the sensor 1. Thus, a compact sensor can be realized, which alleviates the requirement on space close to the region 10 at which the concentration of a substance and/or molecule is to be determined. Light emitting diodes are also advantageous since the intensities, wavelengths, and time responses are controllable. Light emitting diodes can emit at various ranges, which generally extend from 390 nm to 1650 nm although not any wavelength of this range may be achieved.

Further, light emitting diodes have a rather long lifetime as compared to most common light sources and the wavelength range at which they emit can accurately be selected. In particular, the wavelength of the light source has to be selected with respect to the photoluminescent molecules employed for the detection. In general, the absorption range for most photoluminescent molecules extends from 350 to 800 nm. However, each particular photoluminescent molecule has its own absorption band and the appropriate wavelength of the light source depends accordingly. As an example, oxyphors have an absorption maximum near 636 nm, which corresponds to the wavelength required to excite the photoluminescent molecules. Thus, a light source 101 emitting at this wavelength is needed. Such a light source can be commercially available light emitting diodes, like the type Toshiba LED lamp TLSU1102 which has an emission peak at 20 mA of 636 nm with intensity of 200 Mcd. A person skilled in the art would understand that other light emitting diodes or light sources capable of emitting light in the range of 610-655 nm would be suitable to excite oxyphors.

The sensitivity of the photo-detector 104 of the implantable sensor 1 depends on the wavelength range at which the photoluminescent molecules 103 emit. Generally, the range of wavelength at which photoluminescent molecules emit extend from 350 to 1800 nm, in particular in the range of 350-800 nm. Thus, the photo-detector 104 shall be sensitive in this range of wavelength. As the wavelength at which a selected photoluminescent molecules emit is known, the sensitivity of the photo-detector can be selected accordingly. As an example, oxyphors emit light at about 800 nm, which corresponds to near infrared. In this case, the photo-detector may be a commercially available planar InGaAs photodiode, which is sensitive to red and infrared light, i.e. in the spectral range of 800-1800 nm. Such a detector has a response time of up to 120 MHz, which is sufficiently rapid for measuring photoluminescent lifetimes and sufficiently quantitative for steady state measurements of intensities as well. The measurements of lifetimes and intensities will be described in more details later. In particular, the photo-detector may be one of the group comprised of a pn photodiode, a pin photodiode, a Schottky photodiode, an avalanche photodiode, a silicon photodiode, a planar InGaAs photodiode, a SiGe-based optoelectronic circuit and a InGaN/GaN multiple quantum well pn junction.

The photoluminescent molecules 103 should be selected in accordance with the substances and/or molecules for which the concentration has to be determined. The sensor 1 may be adapted to detect several kinds of substances and/or molecules comprised in the region 10 at the condition that these molecules can diffuse through the carrier 102 and react with photoluminescent molecules such that the characteristics of the light emitted by the photoluminescent molecules is altered. In particular, the sensor 1 may be adapted to determine the concentration of oxygen and/or the concentration of carbon dioxide in the region 10 of a patient. In another embodiment, the sensor 1 may be adapted to determine the concentration of molecules representative of the pH-value of the region 10, thereby a sensor for determining the pH-value of a region 10 can be achieved. Further, the molecule and/or substance for which the concentration is to be determined may be one of the group comprised of glucose, urea, ammonia, lactose, hormones and insulin. A list of photoluminescent molecules with which glucose concentrations and pH-values may be determined is given later.

The molecules and/or substances for which the concentration is to be determined preferably are gas molecules since the diffusion of gas molecules through the carrier is facilitated as compared to other types of molecules. However, the sensor would also function with liquid substances and/or molecules.

The photoluminescent molecules may be fluorescent molecules or phosphorescent molecules. Although it would be preferable to use fluorescent molecules as they are generally more stable over time, it is preferable to use phosphorescent molecules as phosphorescence leads to longer timescale, which then facilitates the design of the electronics in the sensor.

One of the requirements on the photoluminescent molecules is that they shall react with the molecules and/or substances for which the concentration has to be determined in such a manner that the characteristics of the light that they emit is altered in accordance with the concentration of the molecules and/or substances to be determined. The photoluminescent molecules preferably have a long longevity of luminescence, high quantum yield, good chemical stability in implanted environment and high selectivity to the molecules and/or substances for which the concentration is to be determined. The photoluminescent molecules shall be stable enough such that the intensity of the light that they emit remains sufficient over a long period of time, e.g. ten years. Further, if the sensor is designed to determine the concentration of oxygen in blood, it is preferable that the photoluminescent molecules are not sensitive to other gases than oxygen such as carbon monoxide which can also be found in blood. The photoluminescent molecules shall also be easily bounded to the material of the carrier, i.e. they should be compatible with the characteristics of the carrier.

The photoluminescent molecules 103 may e.g. be immobilized by means of covalent bonding with the material of the carrier 102. This can be done by a wide number of possible chemical synthetic pathways. In an embodiment, the photoluminescent molecules are bounded to the carrier by physisorption or chemisorption to an immobile filler comprised in the carrier, which is especially useful for silicone rubber which normally contains high surface area active silica as a reinforcing filler. In another embodiment, if the photoluminescent molecules are ions, the molecules can be ionically bonded to the carrier. In another embodiment, the photoluminescent molecules are covalently bonded to the carrier, especially as a pendant substitution group to the polymer used as a carrier. In yet a further embodiment, the photoluminescent molecules are immobilized by size immobilization which prevents large molecules from diffusing in the carrier.

In the case of pH, glucose (monosaccharides) and CO₂ sensitive photoluminescent molecules, a hydrophilic substrate coated by the carrier in which the photoluminescent molecules are embedded may be used. Alternatively, a hydrophilic material could be used for the carrier.

Pyrene and various pyrene derivatives are e.g. well suitable for a carrier made of silicone rubber.

In another embodiment, two different types of photoluminescent molecules may be embedded in the carrier such that concentrations of two different types of molecules and/or substances may be determined. The two different types of photoluminescent molecules would then preferably emit at two different wavelengths and a first analysis of the wavelength spectrum, based on the signals output by the photo-detector, would indicate which of the signals refer to which type of photoluminescent molecules.

Photoluminescent molecules that may be used to determined the concentration of oxygen are molecules comprised within the group of pyrene, pyrene derivatives (vinylpyrene, methoxypyrene), a polyaromatic carrier, an ionic probe (ruthenium trisbypyridine) and Pd-tetra (4-carboxyphenyl)benzoporphin (Oxyphor). Additional examples are naphthalene derivatives (e.g. 2-dimethylamino-6-lauroylnaphthalene); polypyridyl complexes of transition metals, particularly Ruthenium, Osmium, or Rhenium containing fluorescent molecules {e.g. Ru(bipy)(3)(2+) (tris(2,2′-bipyridyl)ruthenium(II) chloride hexahydrate) and Ru(phen)(3)(2+) (tris(1,10-phenanthroline)ruthenium(II) chloride hydrate)}; fullerenes (C60 and C70); decacyclene; perylene and perylene derivatives (e.g.—perylene dibutyrate); and metalloporphines especially Pt(II), Zn(II) and Pd(II) variants.

Photoluminescent molecules that may be used to determined the concentration of glucose are boronic acid containing fluorophores (e.g. 4′-dimethylaminostilbene-4-boronic acid; 4′-cyanostilbene-4-boronic acid; 4-amino-3-fluorophenylboronic acid; and 4-carboxy-3-fluorophenylboronic acid).

Photoluminescent molecules that may be used to determined the PH-value of the region are pyrene derivatives (e.g. hydroxypyrene sulfonic acid; aminopyrene; N-(3-Pyrene)succinimidothioethanol); β-methylumbelliferone; fluorescien derivatives (e.g. Carboxynaphthofluorescein; 5,6-carboxyfluorescein; fluoresceinamine; bis-carboxyethylcarboxyfluorescein) cyanine derivatives (e.g. benzothiazolium trimethine cyanine), seminaphthorhodafluor (SNARE) and carboxyseminaphthofluorescein (SNAFL) derivatives.

In further embodiments, the concentration of the photoluminescent molecules 103 is comprised in the range of 20 ppb to 2% w/w, preferentially in the range of 0.1-500 ppm, and more preferentially at approximately 5 ppm.

Generally, the principle of the sensor 1 is based on the deactivation or quenching of the luminescence of the photoluminescent molecules by the molecules and/or substances for which the concentration has to be determined, called quenchers in the following. The quenchers, e.g. oxygen molecules, are capable of deactivating the luminescence of the photoluminescent molecules, i.e. that excited photoluminescent molecules do not give off light because of the presence of and the reaction with quenchers. The effect of the quenching is proportional to the amount of quenchers. The deactivation process is conventionally modeled by the Stern-Volmer relationship, which is expressed as follows:

I(t)=I(0)×exp(−k_{obs}×t) (1)

where I(t) is the transient intensity of the luminescence at time t, I(0) is the intensity of the luminescence at time t=0, t is the time and k_{obs} is the empirically observed pseudo-first order rate constant characteristic of the decay rate of the luminescence of the photoluminescent molecules in presence of quenchers.

The coefficient k_{obs} can be expressed as a function of the first order decay rate constant k₁ representative of the decay rate of the luminescence of the photoluminescent molecules without the presence of quenchers, the second order rate constant of quenching k_{g} representative of the rate at which the photoluminescent molecules are quenched by the quenchers and [Q] the concentration of quenchers, as follows:

k_{obs}=k_{q}×[Q]+k₁ (2)

By integration over time of the Stern-Volmer relationship (1) and using equation (2), the concentration of quenchers can be expressed as:

$$[ Q ] = \frac{{k}_{1}}{{k}_{q}} \times ( \frac{{I}_{0}}{{I}_{f}} - 1 ) \qquad ( 3 )$$

where I_{f} is the steady state intensity of luminescence. Equation 3 shows that there is a relationship between the steady state intensity and the concentration of the quenchers, from which relationship the concentration of the molecules and/or substances can be determined. Thus, the measurement of the intensity of the light emitted by the photoluminescent molecules results in the concentration of the molecules and/or substances 11 in the region 10 of the patient.

Starting from equations (1) and (2), the transient intensity of the luminescence can also be expressed as:

$$ln ( \frac{I ( 0 )}{I ( t )} ) = ( {k}_{q} \times [ Q ] + {k}_{1} ) \times t \qquad ( 4 )$$

Thus, time-resolved measurement showing the time-resolved decay of the luminescence as a function of time enables also to determine the concentration of quenchers. In practice, the natural logarithm of intensity is plotted as a function of time and the concentration of quenchers is extracted from the slope of the resulting straight line. Time-resolved measurements are also called lifetime measurements since this type of measurements is a way of measuring the lifetime of the luminescence, i.e. the time it takes for the luminescence to decay to a minimal value (in principle to a “zero” value). In other words, the lifetime represents the time it takes for extinction of the luminescence of the photoluminescent molecules. Such measurements are advantageous since they are not dependent on the amount of photoluminescent molecules at the time of the measurement. Two different alternatives for measuring lifetime will be described later.

The signals output by the photo-detector may also be analyzed to determine the wavelength of the light emitted by the photoluminescent molecules. When the sensor is employed to determine the concentration of a substance such as oxygen in blood, the wavelength of the light emitted by the photoluminescent molecules will generally be the same and not depend on the amount of oxygen molecules in blood. However, the sensor may be used to determine the PH-value of the region 10. In this case, the photoluminescent molecules are chosen in such a manner that they react with e.g. H₃O⁺ and OH⁻ molecules of the region. As an example, aminopyrene can be used as a photoluminescent molecule. The amino-group of aminopyrene molecules has the ability to react with hydrogen such that it becomes negatively charged if the pH-value is negative and positively charged if the PH-value is positive. As the wavelength at which aminopyrene molecules emit depends on whether they are positively or negatively charged, i.e. depends on the PH-value, the analysis of the wavelength of the light enables to determine the PH-value of the region 10 of the patient.

The carrier 102 is, at least partially, in contact with a region 10 of the patient where the concentration of the molecule and/or substance has to be determined. This region may in principle be any region but in particular, the sensor may be placed at the heart of the patient, in arterial blood, in venous blood or implanted at another organ of the patient. Arranged at such regions, it is probable that the sensor may physically be subjected to little tissue overgrowth. However, the sensor of the present invention would still function since the optical measurement occurs inside the carrier 102 and not directly in the blood or the tissue. In other words, the light emitted from the light source and the light emitted from the photoluminescent molecules do not have to pass through an interface in contact with the region of the patient, which would, otherwise, enhance tissue overgrowth.

In an embodiment, the light source 101 and the photo-detector 104 are hermetically isolated from the region 10 of the patient at which the carrier 102 is arranged.

In the following, some examples of sensors are given for determining the concentration of oxygen, glucose and carbon dioxide in blood. First, three alternatives are presented to implement a sensor for determining the concentration of oxygen in blood.

In a first alternative, the carrier of the sensor is made of poly(dimethylsiloxane), PDMS in short. This material presents the advantage of being flexible, and has suitable biocompatibility and biostability characteristics. In addition, this material has the advantage of being highly permeable to oxygen, which imparts high sensitivity to the oxygen sensor. Decacyclene (DCY in the following), with a concentration in the order of 5 ppm in the carrier, is a suitable probe molecule (or photoluminescent molecule) for sensing the concentration of oxygen since oxygen molecules can quench the fluorescence of DCY very efficiently. However, DCY needs to be solubilized in PDMS, which can e.g. be implemented by introducing tertiary butyl groups to increase solubility. Further, inclusion of a vinyl group enables DCY to be immobilized in the carrier (PDMS) by addition reaction to pendant vinyl groups on a partially vinyl-substituted component of the PDMS precursor. Alternatively, alkyloxy-groups can be included on DCY to enable a condensation reaction that immobilizes DCY with the PDMS. Further, as DCY has an excitation maximum near 380 nm, an inexpensive light source such as a LED emitting in the blue or near UV portion of the spectrum can be used. The light source may be a commercially available blue light laser with an emission maximum at 405 nm. As DCY emits fluorescent light with maximum intensity near 510 nm, a suitable photo-detector is a photodiode which is sensitive to this wavelength and which has a high speed response suitable for lifetime measurements. A possible choice is a silicon-based avalanche photodiode, which provides very high sensitivity in the range of 450-1000 nm and which has a maximum sensitivity in the range of 600-800 nm. Further, a silicon-based avalanche photodiode provides the advantage of being very little sensitive to the light emitted from the blue LED light source, thereby reducing the need for optical filtering to shield the detector from scattered light from the light source.

In a second alternative, the carrier of the sensor may be made of polystyrene (PS), which presents good mechanical properties. Although, the permeability of oxygen in PS is lower than the permeability of oxygen in PDMS, PS presents the advantage that the probe molecules dissolved in PS do not easily diffuse, which means that chemical modifications to immobilize the probe molecules are usually unnecessary. On the other hand, a thin protective coating of PDMS might be useful to protect PS from degradation by biological processes, thereby ensuring a long lifetime to the implanted sensor. In this embodiment, perylene dibutyrate may be used as probe molecules. As these photoluminescent molecules have an excitation maximum near 457 nm and an emission maximum near 512 nm, inexpensive components for the light source, e.g. a blue LED with a light intensity maximum at 460 nm such as the commercially available Opto Technology OTL460A-1-1-46-D-2, and for the photo-detector, such as a silicon-based avalanche photodiode, may be used. Further, perylene dibutyrate has a high degree of photostability as compared to other probe molecule types. The photoluminescent molecules may have a concentration of about 0.1% w/w in the carrier.

In a third alternative, the sensor may be made of Pd(II) octaethylporphyrin-ketone (PdOEPK) dispersed with a concentration in the range of 0.5% w/w (500 ppm) in a PS carrier. Such a sensor presents good photostability, and the photoluminescent molecules have a long lifetime, which facilitates oxygen quantification by determination of luminescent lifetime changes. The sensor is therefore particularly suitable for determining physiological oxygen concentrations. Further, as PdOEPK has excitation maxima around 410 nm and 603 nm and an emission maximum around 790 nm, an inexpensive and highly efficient red LED may be used as a light source and a near infrared detector such as an InGaAS avalanche photo-diode may be used as a photo-detector. The optics of the sensor is also simplified as PdOEPK presents a high quantum yield of luminescence at body temperatures.

Then, an alternative to implement a sensor for determining the concentration of glucose in blood is presented. In this embodiment, the carrier may be made of a hydrophilic polymer to facilitate transport of glucose to the probe molecules immobilized in the carrier or in the carrier film. Films based on polyvinyl alcohol are preferred as they present good biocompatibility and flexibility. Such films have the ability to contain boronic acid-containing fluorophores, such as 4′-dimethylaminostilbene-4-boronic acid (DSTBA), which may be used as photoluminescent molecules in this embodiment. The concentration of the photoluminescent molecules in the carrier may be in the order of 50 ppm. These photoluminescent molecules have an excitation maximum near 340 nm and an emission maximum between 450 and 500 nm depending on the concentration of glucose or other monosaccharides. Thus, a suitable excitation source may be a UV LED with a peak of emission at around 370 nm and a suitable detector may be a silicon-based avalanche photodiode.

Then, an alternative to implement a sensor for determining the concentration of carbon dioxide in blood is presented. In this embodiment, PDMS may be selected as material for the carrier to immobilize the probe molecules since PDMS permeable to carbon dioxide but impermeable to ions, thus minimizing interference by e.g. Cl ions and/or pH effects. PDMS is biostable and biocompatible, thus suitable for long term implant. 1-hydroxypyrene-3,6,8-trisulfonate (HPTS), HPTS in the following, may be used as photoluminescent molecules since its fluorescence characteristics are correlated to the concentration of carbon dioxide. HTPS has an excitation maximum near 470 nm and may therefore be excited by a blue LED having a light intensity maximum at 460 nm, such as the commercially available “Opto Technology OTL460A-1-1-46-D-2”. HTPS has an emission maximum near 510 nm, which may be detected by a silicon-based avalanche photodiode. Further, HTPS presents the advantage of being very little affected by oxygen or other gases found in significant concentrations in a physiological environment. The concentration of HTPS in the carrier may be in the order of 500 ppm.

With reference to FIG. 2, another aspect of the present invention is presented.

FIG. 2 shows a medical lead according to an embodiment of the present invention. The medical lead typically comprises an implantable sensor 1 such as the sensor described above. The medical lead 2 shown in FIG. 2 is an elongated lead in which a depression is formed. The lead would preferably be cylindrical with a circular or elliptic section but the lead may also have other types of sections. The depression extends only on a short portion of the lead 2. Photoluminescent molecules 103 embedded in the carrier 102 are placed inside the depression. In the embodiment shown in FIG. 2, the light source 101 and the photo-detector 104 are also arranged inside the depression. However, as mentioned above for the implantable sensor 1 described with reference to FIG. 1, the light source 101 and the photo-detector 104 may be arranged at a distance from the carrier and optically connected to the carrier by means of optical fibers. The configuration shown in FIG. 2 is particularly well adapted for determining the concentration of a substance and/or molecule comprised in the blood or in the tissue of a patient. The medical lead could be arranged in an artery or a vein but would preferably be arranged at the heart of a patient where there is a high blood flow. The medical lead therefore comprises a screw 202 used to attach the medical lead to the heart.

With reference to FIG. 3, another aspect of the present invention is presented.

FIG. 3 shows an implantable device 3 such as a pacemaker implantable cardioverter-defibrillator (ICD) or similar cardiac simulation device according to an embodiment of the present invention. The implantable device 3 is connected to a medical lead similar to that described with reference to FIG. 2, i.e. a medical lead comprising an implantable sensor similar to that described with reference to FIG. 1. Only some of the features of the sensor are represented in FIG. 3. However, a sensor such as any one of the sensors described in accordance with the embodiments cited above could be used. The implantable device shown in FIG. 3 further comprises a blood constituent determining device 301 adapted to determine the concentration of the quenchers based on the signals output from the photo-detector 104. The determination may be based on the above described equations 3 and 4.

The implantable device 3 may also comprise a pulse-generating means connected to the light source 101 and the constituent determining device 301 such that light pulses may be generated to perform time-resolved measurements, such as those explained with reference to equation 4. For example, a 4 μs light pulse may be generated to excite the photoluminescent molecules 103 arranged in the carrier 102. Measurements of the intensity at different time points like 30, 50, 120, 180, 240, 420 and 2500 μs after the generation of the light pulse may be performed to calculate the characteristic lifetime of the photoluminescent molecules 103 and to determine the concentration of the quenchers in the region 10 in accordance with equation (4).

The implantable device may also comprise a frequency modulating means 303 connected to the light source 101 and the constituent determining device 301 such that an array of harmonics (with for instance 100-200 frequencies) is used to modulate the light source 101. Using such a technique, lifetimes in the range of 10-3000 μs may be measured.

The implantable device may also comprise an amplifier 304 adapted to amplify the signals output from the photo-detector 104.

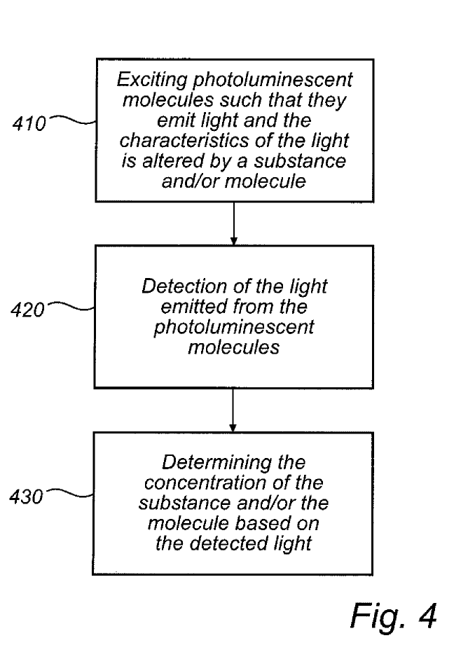

With reference to FIG. 4, another aspect of the present invention is presented.

FIG. 4 shows a block diagram of a method for determining a concentration of a substance and/or molecule in blood or tissue of a patient according to an embodiment of the present invention. In this method, a first step (step 410) consists of, during measurements sessions, exciting photoluminescent molecules embedded in a carrier selectively permeable to the substance and/or molecule such that the photoluminescent molecules emit light. In a second step, step 420, the light emitted from the photoluminescent molecules is detected and in a third step, step 430, the concentration of the substance and/or the molecule is determined based on characteristics of the detected light.

The present invention is applicable in implantable devices for determining a concentration of a substance and/or molecule comprised in the blood composition or in the tissue of a patient. Typically, the present invention is applicable to determine the concentration of oxygen in blood or the saturation of oxygen in blood for patient having a pacemaker.

Although the invention above has been described in connection with preferred embodiments of the invention, it will be evident for a person skilled in the art that several modifications are conceivable without departing from the scope of the invention as defined by the following claims.

I claim as my invention:

1.-61. (canceled)

62. A medical lead comprising:
a lead body configured for in vivo implantation in a subject;
a sensor carried by said lead body that determines a concentration of a blood constituent, selected from the group consisting of a substance and a molecule, in blood or tissue of the patient;
said sensor comprising at least one light source that is activatable, in a measurement session, to emit light at least at a first wavelength, at least one photodetector, and a carrier having at least one type of photoluminescent molecules embedded therein that are selectively permeable to said constituent, said at least one light source and said at least one photodetector being optically coupled to said carrier;
said lead body being configured to place said sensor, when said lead body is implanted in the patient, at a sensor site selected from the group consisting of in the heart of the patient, in arterial blood of the patient, in venous blood of the patient, and in tissue of a blood-containing organ of the patient, to cause said constituent to interact with said photoluminescent molecules in said carrier;
said at least one type of photoluminescent molecules being selected to be excitable by said light emitted by said at least one light source to emit photoluminescent light and to react with said constituent in said carrier to alter characteristics of said photoluminescent light;
said at least one photodetector detecting the photoluminescent light emitted by said photoluminescent molecules and emitting a detector output signal dependent thereon that represents a concentration of said constituent in said blood; and
said carrier being configured to cause emission of said photoluminescent light from said photoluminescent molecules, and detection of said photoluminescent light by said detector, to occur within said carrier.

63. A medical lead as claimed in claim 62 wherein said at least one type of photoluminescent molecules react with a constituent that is representative of the pH-value at said sensor site.

64. A medical lead as claimed in claim 62 wherein said at least one type of photoluminescent molecule is selected from the group consisting of oxygen, carbon dioxide, glucose, urea, ammonia, lactose, hormones and insulin.

65. A medical lead as claimed in claim 62 wherein said at least one type of photoluminescent molecules are phosphorescent molecules.

66. A medical lead as claimed in claim 62 wherein said at least one light source and said at least one photodetector and hermetically isolated from said sensor site.

67. A medical lead as claimed in claim 62 wherein said characteristic is selected from the group consisting of intensity, lifetime and wavelength of said photoluminescent light.

68. An implantable device comprising:
a lead body configured for in vivo implantation in a subject;
a sensor carried by said lead body that determines a concentration of a blood constituent, selected from the group consisting of a substance and a molecule, in blood or tissue of the patient;
said sensor comprising at least one light source that is activatable, in a measurement session, to emit light at least at a first wavelength, at least one photodetector, and a carrier having at least one type of photoluminescent molecules embedded therein that are selectively permeable to said constituent, said at least one light source and said at least one photodetector being optically coupled to said carrier;
said lead body being configured to place said sensor, when said lead body is implanted in the patient, at a sensor site selected from the group consisting of in the heart of the patient, in arterial blood of the patient, in venous blood of the patient, and in tissue of a blood-containing organ of the patient, to cause said constituent to interact with said photoluminescent molecules in said carrier;
said at least one type of photoluminescent molecules being selected to be excitable by said light emitted by said at least one light source to emit photoluminescent light and to react with said constituent in said carrier to alter characteristics of said photoluminescent light;
said at least one photodetector detecting the photoluminescent light emitted by said photoluminescent molecules and emitting a detector output signal dependent thereon that represents a concentration of said constituent in said blood;
said carrier being configured to cause emission of said photoluminescent light from said photoluminescent molecules, and detection of said photoluminescent light by said detector, to occur within said carrier; and
an evaluation unit supplied with said signal from said detector output that identifies the concentration of said constituent from said signal.

69. An implantable device as claimed in claim 68 wherein said at least one type of photoluminescent molecules react with a constituent that is representative of the pH-value at said sensor site.

70. An implantable device as claimed in claim 68 wherein said at least one type of photoluminescent molecule is selected from the group consisting of oxygen, carbon dioxide, glucose, urea, ammonia, lactose, hormones and insulin.

71. An implantable device as claimed in claim 68 wherein said at least one type of photoluminescent molecules are phosphorescent molecules.

72. An implantable device as claimed in claim 68 wherein said at least one light source and said at least one photodetector and hermetically isolated from said sensor site.

73. An implantable device as claimed in claim 68 wherein said characteristic is selected from the group consisting of intensity, lifetime and wavelength of said photoluminescent light.

74. An implantable device as claimed in claim 68 comprising an amplifier that amplifies said output signal from said at least one photodetector prior to said output signal being supplied to said evaluation device.

75. An implantable device as claimed in claim 68 comprising a pulse generator connected to said at least one light source that operates said at least one light source to emit said light as light pulses, and wherein said evaluation unit is configured to control said at least one photodetector and said pulse generator to implement time-resolved measurements to determine a lifetime of said photoluminescent light.

76. An implantable device as claimed in claim 68 comprising a frequency modulator connected to said at least one light source that operates said at least one light source to emit said light as light pulses, and wherein said evaluation unit is configured to control said at least one photodetector and said frequency modulator to implement time-resolved measurements to determine a frequency modulation of said photoluminescent light.

77. A method for determining a concentration of a constituent in blood of a patient, said method comprising the steps of:
providing a lead body configured for in vivo implantation in a subject with a sensor carried by said lead body that determines a concentration of a blood constituent, selected from the group consisting of a substance and a molecule, in blood or tissue of the patient, said sensor comprising at least one light source that is activatable, in a measurement session, to emit light at least at a first wavelength, at least one photodetector, and a carrier having at least one type of photoluminescent molecules embedded therein that are selectively permeable to said constituent, said at least one light source and said at least one photodetector being optically coupled to said carrier;
implanting said lead body to place said sensor at a sensor site selected from the group consisting of in the heart of the patient, in arterial blood of the patient, in venous blood of the patient, and in tissue of a blood-containing organ of the patient, to cause said constituent to interact with said photoluminescent molecules in said carrier;
selecting said at least one type of photoluminescent molecules to be excitable by said light emitted by said at least one light source to emit photoluminescent light and to react with said constituent in said carrier to alter characteristics of said photoluminescent light;
detecting the photoluminescent light emitted by said photoluminescent molecules with said at least one photodetector, and emitting a detector output signal therefrom dependent thereon that represents a concentration of said constituent in said blood; and
configuring said carrier to cause emission of said photoluminescent light from said photoluminescent molecules, and detection of said photoluminescent light by said detector, to occur within said carrier.
